(19) United States
(12) Patent Application Publication
(10) Pub. No.: US 2015/0329887 A1
(43) Pub. Date: Nov. 19, 2015

(54) INTEGRATED BIOREFINERY

(71) Applicant: MYRIANT CORPORATION, Woburn, MA (US)

(72) Inventors: Bin Wang, Wobrun, MA (US); Zachary Wilson, Quincy, MA (US); Ramnik Singh, Winchester, MA (US)

(73) Assignee: MYRIANT CORPORATION, Quincy, MA (US)

(21) Appl. No.: 14/368,719

(22) PCT Filed: Dec. 20, 2012
(86) PCT No.: PCT/US2012/070902

§ 371 Date: Jun. 25, 2014

Related U.S. Application Data
(60) Provisional application No. 61/631,268, filed on Dec. 30, 2011.

(51) Int. Cl. C12P 19/14; C12P 19/02

(52) U.S. Cl. CPC C12P 19/14; C12P 19/02; C12P 2201/00

(57) ABSTRACT

This invention relates to the operation of a biorefinery for manufacturing either biofuels or renewable chemical feedstock using lignocellulosic biomass as a source of carbon. The present invention provides a cost-effective process for pretreating lignocellulosic biomass in the recovery of fermentable sugars. More specifically, the present invention describes an integrated approach for efficiently recovering and using six-carbon and five-carbon sugars along with value-added oligosaccharides such as xylooligosaccahrides from lignocellulosic biomass so that the cost of manufacturing biofuels and renewable chemical feedstock is substantially lowered.

### CROSS-REFERENCE TO RELATED APPLICATION

This application claims the priority of the U.S. Provisional Application Ser. No. 61/631,268, filed on Dec. 30, 2011.

### FIELD OF THE INVENTION

The present invention is in the field of producing biofuels and renewable chemical feedstock using biocatalysts that have been genetically engineered to increase their ability to use renewable carbon resources. More specifically, the present invention provides a process for operating an integrated biorefinery utilizing lignocellulosic biomass in the production of biofuels and renewable chemical feedstock along with value-added xylooligosaccharides which are useful as nutraceuticals.

### BACKGROUND OF THE INVENTION

There has been growing interest in developing alternate transportation fuels and chemical feedstock using renewable resources. The term alternate transportation fuel also known as biofuels as used in this invention refers to the fuel alcohols including ethanol and n-butanol produced by microbial fermentation using renewable biological feedstock. The term renewable chemical feedstock also known as renewable biochemicals or renewable chemicals as used in this present invention refers to the chemicals that are produced from carbon sources derived from biomass through microbial fermentation as opposed to the same type of chemicals manufactured through chemical reactions using petrochemical feedstock.

A 2004 U.S. Department of Energy report entitled “Top value added chemicals from biomass” has identified twelve building block chemicals that can be produced from renewable feedstock. The twelve sugar-based building block chemicals are 1,4-diacids (succinic, fumaric and maleic), 2,5-furan dicarboxylic acid, 3-hydroxy propionic acid, aspartic acid, glucaric acid, glutamic acid, itaconic acid, levulinic acid, 3-hydroxybutyrolactone, glycerol, sorbitol, and xylitol/arabinitol. Building block chemicals are molecules with multiple functional groups that possess the potential to be transformed into new families of useful molecules that are suitable for chemical synthesis of polymers. Thus, these twelve building blocks can be subsequently converted to a number of high-value bio-based chemicals or materials.

In recent years, the efficiency of microorganisms for producing monomeric chemical compounds suitable for industrial usage has been significantly increased through genetic manipulations. However, at present the cost of producing industrial chemicals through biological fermentative process is still very high; the cost associated with renewable feedstock contributes significantly to the manufacturing process.

First generation carbohydrate materials for the production of biofuel and the renewable chemical feedstock come from cereal grains and sugar crops that are also source of human and animal food. The sugar crops such as sugar cane and sugar beet contain readily fermentable sucrose. The cereal crops like maize and wheat contain starch as their primary carbohydrate material and require pre-hydrolysis prior to sugar fermentation. However, a continued use of first generation feedstock in the production of biofuel and renewable chemical feedstock is not sustainable in the long run due to the concerns about human food security and land-use issues. There has been effort to develop second generation feedstock which would reduce the cost of production of biofuel and renewable chemicals.

The term second generation feedstock as used in this present invention refers to non-food lignocellulosic biomass. Lignocellulose is the most abundant form of renewable carbon on the earth. Lignocellulosic biomass available for biofuel production and renewable chemical feedstock manufacturing can be grouped under two categories. (1) Biowaste material including straws, corn residues (stover, fibers, and cobs), woody wastes/chipping, forestry residues, old paper/cardboard, bagasse, spent grain, municipal solid waste, agricultural residues (oil seed pulp, sugar beet pulp, etc.); (2) Energy crops including but not limited to short rotation crops such as basket willow (Salix viminalis), energy grass (Miscanthus giganteus), alfalfa (Medicago sativa), switch grass (Panicum vigratum), reed canary grass (Arundo donax), rye grass etc.

A recent report from U.S. Departmetnt of Energy entitled “U.S. Billion-Ton Update—Biomass supply for a Bioenergy and Bioproducts Industry” has projected that the US would have between 1.1 and 1.6 billion tons of sustainable biomass available for industrial bio-processing by 2030. The challenge in front of the bio-processing industry is to recover the fermentable sugars from the lignocellulosic biomass in a cost-effective way.

The cost of fermentation process for producing biofuels and industrial chemicals can be significantly reduced by using lignocellulosic biomass as the source of carbon in the fermentation process. Lignocellulosic biomass consists of roughly 40-50% of hexose sugars and 10-30% of pentose sugars. The hexose sugars are known in the art as C6 sugars. The pentose sugars are known in the art as C5 sugars. When hydrolyzed, the lignocellulosic materials yield a mixture of sugars that includes glucose, xylose, arabinose, mannose and galactose. However, most of the biocatalysts currently used in the fermentation processes for the production of biofuel and industrial chemicals utilize pure glucose as a source of carbon for their growth and metabolism. For example, the E. coli strain useful in the fermentative production of lactic acid described in U.S. Pat. No. 7,223,567 uses a rich medium supplemented with glucose as the source of carbon. The E. coli strain KJ122 useful for the production of succinic acid described by Jantama et al (2008a; 2008b) and in the published PCT Patent Applications Nos. WO/2008/021141A2 and WO2010/115067A2 requires a minimal medium supplemented with glucose.

The ability of the microorganism to use multiple sugars simultaneously is limited by the existence of certain biochemical regulatory systems. These biochemical regulatory systems within the microbial cells have a genetic basis. At present the industrial microorganisms are grown in a medium containing glucose or sucrose as the source of carbon. The presence of glucose in the growth medium suppresses the use of other sugars in E. coli and other species of industrial microorganisms. The consumption of other sugars such as xylose, a pentose sugar, by these microorganisms is initiated only after glucose in the growth medium has been fully consumed. This phenomenon related to carbon utilization in industrial microorganisms is referred to as catabolite repression or diauxic growth. A method to make the microorganisms co-utilize the different sugars such as C5 and C6 sugars through a relief of catabolite repression during the production of industrial chemicals in a commercial scale would be critical to lowering the cost of industrial chemicals produced by fermentation. Alternately, the C5 and C6 sugars from the lignocellulosic hydrolysate can be recovered in separate streams and subsequently fed to the biocatalysts at different times in order to maximize the use of both C5 and C6 fermentable sugars recovered from lignocellulosic biomass. Thus by means of utilizing both C5 and C6 sugars recovered from the lignocellulosic feedstock, the cost of manufacturing biofuel and renewable chemical feedstock using lignocellulosic biomass can be significantly reduced. Yet another approach to reduce the cost of manufacturing biofuel and renewable chemical feedstock using lignocellulosic biomass is to recover certain value-added chemicals from lignocellulosic biomass besides recovering fermentable sugars from lignocellulosic biomass. For example, value-added nutritional dietary fibers can be recovered from the lignocellulosic hydrolysate besides fermentable sugars.

Dietary fibers are complex carbohydrates resistant to digestion by human digestive enzymes. They can be classified as insoluble and soluble fibers. The fibers that are naturally present in the food are referred as insoluble dietary fibers. Diets high in fiber-rich carbohydrates can improve glucose and insulin concentration and also contribute to a decrease in blood lipids for people with Type II diabetes. However, the levels of fiber required to induce these beneficial effects are high (up to 35 g/day) which may be difficult for people to achieve only with the insoluble fibers naturally present in the food. For this reason, the development of the soluble dietary fiber (SDF), has gained increasing importance.

SDFs include pectin, beta-glucans, fructans, oligosaccharides, some hemicelluloses, guar and gums. For instance, polydextrose is a non-digestible synthetic polymer of glucose. It is used as a food ingredient and classified as soluble fiber by the U.S. Food and Drug Administration (FDA). It is frequently used in place of sugar as a way of reducing calories. It is a multi-purpose food ingredient synthesized from dextrose (glucose), plus about 10 percent sorbitol and 1 percent citric acid. U. S. FDA approved it in 1981.

Non-digestible oligosaccharides are low molecular weight carbohydrates of intermediate in nature between simple sugars and polysaccharides. Their industrial applications have rapidly increased in the last few years. Functional oligosaccharides have been reported to have effects on the coronary heart disease risk reduction, weight control, glucose control for diabetic patients, decreasing serum total cholesterol and low-density lipoprotein cholesterol concentrations etc.

Xylooligosaccharides (XOS) is a high value product often used as a prebiotic substance or a functional food ingredient (Aachary and Prapulla, 2011). XOS can be obtained from breaking down hemicellulose (xylan) present in the lignocellulosic materials. XOS typically contain 2-7 xylose molecules. It is 0.4 times as sweet as sucrose and provides an increased viscosity that leads to improved mouth-feel. XOS possesses a high moisture-retaining capacity and a low water activity, which prevent excessive drying of a food and help to control microbial growth. Furthermore, XOS is more stable at low pH and high temperatures than other oligosaccharides. It cannot be used by mouth flora, and hence is useful as a low-cariogenic sugar substitute. XOS cannot be metabolized by the human digestive system, and is hence suitable for use in sweet, low-calorie anti-obesity diets, and for consumption by individuals with diabetes.

As a dietary fiber, XOS is used for constipation prevention. The indigestible XOS can reach the large bowel where it is fermented by intestinal flora into mainly short-chain fatty acids (SCFA). The production of SCFA is related to a number of health benefits including bowel function, calcium absorption, lipid metabolism, and reduction of the risk of colon cancer. XOS helps to suppress blood cholesterol levels, especially LDL-cholesterol, by binding to bile acids, which are made of cholesterol, in the gastrointestinal tract and carrying them out of the body as waste. It also inhibits lipid absorption in the digestive tract. In addition, fermentation of SDF in the colon generates propionic acid which can suppress the synthesis of cholesterol. XOS's ability to increase the population of Bifidobacterium is the best among all currently available oligosaccharides. Recent reports indicate that Bifidobacterium is useful in promoting gastrointestinal health, preventing colonization of potentially pathogenic and putrefactive bacteria, and enhancing the immune system. It was also found that XOS exhibited 70% inhibition of DNA synthesis of human leukemia HL 60 cells, thus having potential use as cancer cell apoptosis inducers. Oligosaccharides comprising xylose and other sugar residues such as arabinose are also reported to be beneficial to human health. For example, SDF comprising arabinoxylan has been reported to reduce the postprandial glucose and improves metabolic control in people with Type II diabetes.

There is a growing market for oligosaccharides as sweeteners, prebiotics, anticariogenic compounds, and immunostimulating agents in both the food and pharmaceutical industries. For example, by introducing Milky Way II, Mars became the first candy manufacturer to try to gain or retain calorie- and fat-conscious customers. Some of the sugar in Milky Way II is replaced with polydextrose, a low-calorie carbohydrate very similar with cellulose-derived oligosaccharides known as cello oligosaccharide (COS). The resulting candy bar is 25 percent lower in total calories and has 50 percent fewer calories from fat than the original Milky Way.

Xylooligosaccharide (XOS) is a new oligosaccharide that is the best among all currently available oligosaccharides in its ability to increase the population of Bifidobacterium. The production and consumption of XOS has experienced a fast growth in recent years. In Japan, from 1993 to 2002, the average annual increase in market demand for XOS was over 76%. If the consumption in other Asian countries (China, Korea, etc.) and in Europe, as well as the potential market in the North America is taken into account, it is foreseeable that a high demand for XOS will be sustained. As of 2011, there are more than 200 XOS products sold in China at the selling price between $10/kg to $50/kg depending on the purity of the XOS preparation.

XOS have been shown to function as prebiotics in the human body. Prebiotics are typically oligosaccharides that promote the growth of healthy microflora, including Bifidobacterium and Lactobacilli, in the human intestinal tract. These bacteria assist in the breakdown of food and uptake of essential nutrients. In addition, XOS offer an array of other dietary benefits to consumers including fiber like properties, reducing cholesterol, improving uptake of calcium, and acting as antioxidants

### BRIEF SUMMARY OF THE INVENTION

This present invention provides an integrated process for the production of xylooligomers useful in human nutrition and fermentable sugars useful in the biorefineries manufacturing biofuels and renewable chemicals. The integrated process according to the present invention utilizes lignocellulosic biomass derived from agricultural wastes, forestry wastes, municipal solid wastes and energy crops as the source of carbon in the fermentation process by the biocatalysts. The sugars derived from lignocellulosic biomass are used by biocatalysts in the fermentative production of biofuel and renewable chemicals. The biocatalysts suitable for the use in the biorefineries according to the present invention include naturally occurring as well as genetically-modified yeast, fungal and bacterial species. The integrated process described in the instant invention allows recovering value-added oligosaccharides from the lignocellulosic biomass besides obtaining fermentable sugars useful in the manufacture of biofuels and chemical feedstock in a biorefinery. The ability to derive value-added chemicals besides the fermentable sugars from lignocellulosic biomass allows a significant cost-reduction in the overall operation of a biorefinery for the production of biofuels and renewable chemical feedstock.

In one embodiment, the present invention provides two-stage hydrolysis process to obtain xylooligosaccharides (XOS) and fermentable sugars form the lignocellulosic biomass. The terms xylose oligomers and xylooligosaccharides are used interchangeably in the present invention. In the first stage hydrolysis process, the lignocellulosic biomass is subjected to a thermal or a thermochemical treatment to achieve depolymerization of the hemicellulose component of lignocellulosic materials. Optionally, the initial thermal or thermochemical treatment is followed by an enzyme treatment to recover the remaining hemicellulose component from the lignocellulosic biomass. The aqueous phase resulting from the first stage hydrolysis and/or enzyme treatment of lignocellulosic biomass is subjected to appropriate fractionation processes to recover XOS and xylose monomer. The XOS resulting from this combined initial thermochemical and enzymatic hydrolytic reactions represents value-added product while the xylose monomer is used as a fermentable sugar in the production of biofuels and renewable chemical feedstock.

In one aspect of the present invention, the lignocellulosic biomass is subjected to delignification process before subjecting it to the first stage hydrolytic process for recovering xylose monomer and XOS.

In a preferred embodiment of the present invention, the lignocellulosic biomass is subjected to mechanical milling operation to achieve a size reduction which would increase the efficiency of infiltration of the chemical reagents used in the subsequent hydrolysis reactions.

Following the enzymatic hydrolysis process for the release of xylose and XOS from hemicellulose, the depolymerization of cellulose component in the lignocellulosic biomass is accomplished through enzyme digestion leading to the production of fermentable glucose.

In another embodiment, the present invention provides a method for recovering XOS from a wood pulp derived from lignocellulosic biomass. In one aspect of the present invention, the wood pulp used in the recovery of XOS is at least partially bleached. The partially bleached pulp is combined with an alkaline or caustic solution and subjected to treatment at elevated temperature. The resulting solution is subjected to nanofiltration and the hemicellulose component rejected by the nanofiltration membrane exits the nanofiltration system as a concentrated hemicelluloses stream. The hemicellulose stream is acidified to precipitate the hemicellulose component. The resultant white hemicellulose paste is subjected to hydrolytic reaction to obtain XOS and xylose monomers. In one aspect of the present invention, the hemicelluloses recovered by acid precipitation can be completely hydrolyzed using appropriate hydrolytic enzyme to obtain xylose monomer which can be used as a source of fermentable sugar by the biocatalysts in the manufacture of biofuels and renewable chemical feedstock. In a preferred aspect of the invention, the white hemicelluloses paste is subjected to enzyme hydrolysis with endo-xylanse enzyme to produce XOS.

In yet another preferred embodiment of the present invention, the raw cellulose feedstock may be solubilized prior to pulping. Solubilization is advantageous as it partially decomposes and lowers the molecular weight of the hemicellulose within the lignocellulosic material. Solubilized hemicellulose is more readily removed from cellulose fibers than hemicellulose that has not been solubilized.

In this invention, a robust integrated biorefinery process is proposed to effectively and economically recover both xylose monomer and xylose oligomers along with the fermentable sugars from lignocellulosic biomass. Integrated process improvements have been made to the downstream process trains followed in a biorefinery operation leading to the recovery of XOS which is very much different from the processes known in the art for the recovery of XOS from lignocellulosic biomass. The novel integrated process of the present invention for the recovery of both XOS and fermentable sugars form lignocellulosic biomass offers flexibility, enhances process robustness and maximizes the profitability in the operation of a biorefinery utilizing lignocellulosic biomass as the feedstock.

In yet another embodiment of the present invention, the integrated biorefinery includes process steps for recovering lignin component of the lignocellulosic biomass in a highly pure form leading to further reduction in the cost for the operation of a lignocellulosic biomass-based biorefinery.

### BRIEF DESCRIPTION OF THE DRAWINGS

The following figures are included to illustrate certain aspects of the present invention, and should not be viewed as exclusive embodiments. The subject matter disclosed is capable of considerable modifications, alterations, combinations, and equivalents in form and function, as will occur to those skilled in the art and having the benefit of this disclosure.

FIG. 1. Process for recovering XOS from lignocellulosic biomass. In one pathway, the lignocellulosic biomass is subjected to first hydrolysis reaction in which the lignocellulosic biomass is subjected to thermal or thermochemical treatment. This first hydrolysis reaction is also referred as pretreatment. In the second pathway for recovering XOS form lignocellulosic biomass, the pretreatment (the first hydrolysis) is followed by an enzyme digestion of the lignocellulosic biomass. In the third pathway for XOS recovery from lignocellulosic biomass, the lignocellulosic biomass is directly subjected to enzyme digestion without any thermal or thermochemical hydrolysis step. The aqueous extract derived from the lignocellulosic biomass at the end of each of these three different pathways for XOS recovery is subjected to a variety of downstream processing to obtain XOS in a commercially suitable form.

FIG. 2. Integration of XOS recovery from lignocellulosic material (corn cob grits in this case) with the isolation of monomeric sugars useful in the fermentative production of biofuels and biochemicals. The corn cob grits are subjected to mild acid treatment. The acid-treated cellulosic material is maintained at 140-170° C. for 20-40 minutes followed by enzyme digestion with endo-xylanase at pH 5.0 and 50° C. for 24-48 hours. Subsequent to the enzyme digestion, the corn cob grit suspension is filtered to remove the cellulose component containing C6 sugars and the lignin component of the lignocellulosic biomass. The filtrate contains XOS which is further subjected to ultrafiltration and nanofiltration. The fraction rejected from the nanofiltration step contains XOS which can be further purified by polishing and concentrated through evaporation. The permeate containing monomeric sugars such as xylose and glucose can be used in the fermentative production of other products such as biofuels and biochemicals

FIG. 3. Representative profile of the xylooligosaccharides separated using HPLC equipment. Shown in the figure are the representative peaks for xylotetrose, xylotriose, xylobiose, xylose, fructose, glucose and arabinose. The corresponding numbers in brackets represent the retention time for each of the components.

FIG. 4. Kinetics for release of various xylooligosaccharides during enzymatic digestion with endoxylanase HTec2®. Enzyme digestion was carried out for a period of 48 hours.

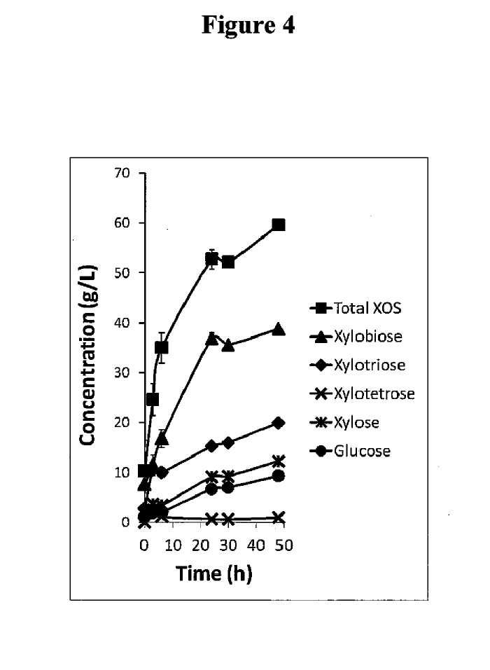

### DETAILED DESCRIPTION OF THE PREFERRED EMBODIMENTS

A number of readily available lignocellulosic biomass can be used in the present invention. It is ideal to use a lignocellulosic biomass material which is known to have significant amount of hemicellulose component so that the recovery of XOS and other desirable SDF is commercially profitable. For example, agricultural crop residues, sugarcane bagasse, hardwoods, corn cobs, barley hulls, brewery spent grains, almond shells, corn stover and corn fiber, rice hulls, flax skive, wheat straw and bamboo have been reported suitable for XOS production and all of these lignocellulosic biomass material are also suitable for the operation of integrated biorefinery of the present invention. Among the various lignocellulosic biomass sources, corn cobs and corn fibers are desirable materials for the operation of the biorefinery of the present invention as both these sources are reported to have high hemicellulose content and are expected to yield commercially significant quantities of XOS. Among the lignocellulosic biomass with high level of hemicelluloses content, the sources with high proportion of xylose in the hemicellulose component is preferred most. Corn cob usually contains about 35% hemicellulose (comprising mostly xylose) and 38% cellulose. Corn fiber from wet milling plant contains about 20% starch, 15% cellulose, and 35% hemicellulose (mostly arabinoxylan).

Hemicelluloses are linear polymers composed of cyclic 5-carbon and 6-carbon sugars. There are five main classes of hemicelluloses, namely galactoglucomannan, arabinoglucuronoxylan, arabinogalactan, glucuronoxylan, and glucomannan. In the native state, hardwood hemicellulose has an average degree of polymerization (DP) of approximately 200, and 80-90% of the principle monomer component is anhydrous D-xylose.

As used in this present invention, the term xylan refers to a naturally occurring polymer of xylose, a 5-carbon sugar. Xylan is also referred as a pentosan.

Cellulose is the main component of wood, contributing 40-50% to the total dry mass. Like hemicelluloses, cellulose is a linear polymer. However, the DP of cellulose is much higher, typically between 1,000 to 10,000, and cellulose chains are composed entirely of anhydrous D-glucose units.

Lignin is a network polymer composed of phenyl-propane monomers, namely p-coumaryl alcohol, coniferyal alcohol, and sinapyl alcohol, which are generally referred to as cinnamyl alcohols, and are commonly known as lignin C9-units. It contributes to approximately 15% to 35% of the dry mass of softwoods, hardwoods, and woody grasses. Lignin is deposited between individual wood fibers and acts as an intercellular adhesive, binding individual wood fibers together.

The key steps in bioconversion of lignocelluloses to fuels are size reduction, pretreatment, hydrolysis and fuel production. Prior to subjecting the lignocellulosic biomass to pretreatment process, the biomass is cleaned and adjusted for size and moisture content. A number of mechanical processes are known in the art to reduce the size of the lignocellulosic biomass to an optimal size so that a maximum recovery of XOS and fermentable sugars is achieved from the lignocellulosic biomass as a result of subsequent thermochemical and enzymatic treatment. For example, knife-milling or hammer-milling can be used to reduce the biomass size.

Aqueous slurry of size-reduced lignocellulose material is utilized as the starting material for this present invention. Water is added to lignocellulosic material in the range of 5% to 30% of total slurry on the weight basis. The appropriate amount of water is dependent on the nature of the lignocellulosic material used and on the type of solubilization technique followed in releasing the hemicellulose component from the lignocellulosic biomass during pretreatment process. The objective of the pretreatment process is to increase porosity of biomass particles in order to increase the accessibility of cellulose and other polysaccharides to the hydrolytic enzymes. However, most pretreatment methods also result in some hydrolysis. The most widely used pretreatment method involves heating the aqueous slurry of lignocellulosic biomass in dilute acid such as 0.9% H₂SO₄. Treatment for as little as one minute at 180° C. in 0.9% H₂SO₄ can result in solubilization of as much as 90% of xylan. The solubilization is presumably associated with two types of chemical reactions: (a) the hydrolysis of xylans to monomeric sugars and oligosaccharides with much higher solubility than intact xylans and (b) the hydrolysis of lignin-xylan or xylan-xylan esters and of acetyl groups on polysaccharides. Since a substantial amount of xylans is thought to be hydrogen bonded to the surface of cellulose microfibrils, the acid pretreatment presumably exposes the cellulose microfibrils to some extent, both by hydrolysis of xylan and also by releasing lignin from indirect association with cellulose via linkage to xylan. Other methods, such as ammonia fiber expansion, cause similar effects.

As used in this invention, the term “solubilization” refers to exposing the lignocellulosic biomass to high pressure and temperature for a specified duration with or without catalyzing additives. In general, solubilization is achieved by a hot-water extraction in a pressurized vessel at an elevated temperature up to about 250° C. at a pH below about 7.0 to yield an aqueous extract containing hemicellulose component. In a preferred embodiment, the lignocellulosic biomass is subjected to a steam explosion. The lignocellulosic biomass is subjected to pressure at temperatures of 100° C. to 250° C. for a period of 2-180 minutes. In a preferred embodiment, the biomass is subjected steam using a steam gun at temperatures of 100° C. to 250° C. for a period of 2-180 minutes. In certain preferred solubilization procedures, acids can be added to enhance the solubilization of hemicelluloses component from the biomass. Acid can be used when the biomass has insufficient acetate residues on the hemicellulose sugars to acidify the mixture sufficiently. Examples of suitable acids include acetic acid, sulfuric acid, nitric acid, hydrochloric acid, phosphoric acid, and carbonic acid. Alkali may also be added during the pretreatment to remove lignin. During the solubilization process using steam gun, the volatiles are vented out at the end of the solubilization, and the aqueous slurry is subjected to separation process by which the aqueous fraction is separated from insoluble phase. The separation of the aqueous and the insoluble phases after solubilization step can be achieved by using any one of the techniques known in the art. For examples an extruder or a centrifuge can be used to separate the aqueous phase from the insoluble phase.

Production of XOS form xylan-rich lignocellulosic materials generally includes chemical methods, enzymatic methods, and a combination of chemical and enzymatic methods. The production of XOS with chemical method can be accomplished by steam, diluted solutions of mineral acids, or alkaline solutions. Extraction of xylan with steam or acid produces large amounts of monosaccharides and their dehydration product. Steam or hydrolytic degradation of xylan, known as autohydrolysis, involves the deacetylation of xylans to produce acetic acid, which hydrolyze the hemicelluloses. This method eliminates the use of corrosive chemicals for the extraction of xylan. However, it requires special equipment that can be operated at high temperatures. The production of XOS with direct enzyme treatment of xylan-containing material is the only suitable method for susceptible materials such as citrus peels. To produce XOS with chemical and enzyme methods, xylan is generally extracted from lignocellulosic materials with hot water, or acid or an alkaline substances such as KOH or NaOH, and extracted xylan is converted to XOS by xylanase enzyme having low exo-xylanase and/or β-xylosidase activity. In contrast to autohydrolysis, the enzyme method is more desirable, because it does not produce undesirable byproducts or high amount of monosaccahrides and does not require special equipment.

The XOS product obtained from lignocellulosic biomass is fractionated via ultrafiltration and nanofiltration using different membranes. Complete removal of xylanase and unhydrolysed xylan is achieved without losing any oligosaccharides having DP 5 or smaller by 10 kDa membrane. After a two-step membrane processing, a permeate containing mostly oligosaccharides is obtained.

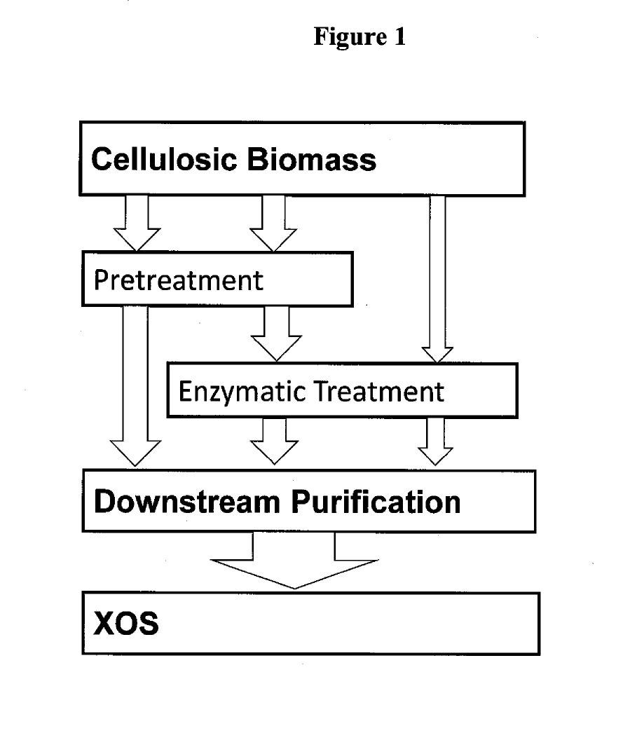

Both route 1 and route 2 in FIG. 1 have advantages and disadvantages. When autohydrolysis is used for XOS production, typically >180° C. is needed for getting a decent XOS yield. A broad range DP of XOS will be present in the broth. Monomer (Xylose) will coexist with long chains oligomers (>10). When chemicals, for example dilute H₂SO₄ is used as catalysts, the reaction is harder to control, and xylose and/or a certain amount of degradation compounds like furfural, HMF or formic acid will appear in hydrolysate. These degradation products along with sulfate will need extra cleaning up efforts. In route 2, endo-xylanase is used for degrading xylan. This enzyme is very helpful to bring DP into suitable range. But addition of endo-xylanase will also increase the production cost. If the pretreatment step is too mild, the enzyme dose has to be large and hydrolysis duration will be longer which brings up contamination concern. If the pretreatment step is harsh, enzyme dose can be kept low, but we will get a large amount of xylose which stands for high yield loss for XOS. The optimal operation conditions are not easy to identify. The compromising point is susceptible to the changes of feedstock and operation facilities.

In order to assure that all hemicelluloses component is extracted from the lignocellulosic biomass, the first thermochemical hydrolysis step can be followed by an enzyme digestion. The list of enzymes that are suitable at this stage includes a variety of endo-xylanase enzymes which are able to release the xylooligosaccharides from the lignocellulosic biomass. At the end of this optional enzymatic treatment, the aqueous phase and the insoluble phase are separated using one or more of the techniques well known in the art. The aqueous phases obtained before and after enzyme treatment can be pooled together and subjected to one or more of the well known separation processes known in the art for recovering XOS. The combined aqueous phase is expected to have a mixture of xylan, XOS and monomeric sugars.

The xylan in the aqueous phase can be broken down into XOS either by acid hydrolysis or enzymatic treatment. It should be kept in mind that the acid treatment is associated with certain potential problems. The yield of XOS is minimal with acid hydrolysis because acid prefers to cleave xylan into individual xylose units and produces several toxic compounds including furfural. Enzymatic treatment of xylan does not produce toxic by-products, but it still produce considerable amount of xylose.

A variety of xylanse enzymes known in the art are useful for obtaining XOS from lignocellulosic biomass according to the present invention. The endoxylanase enzyme suitable for the present invention can be derived from a variety of sources such fungal species such as Neocallimastix frontalis and Neocallimastix patriciarum. An immobilized form of xylanase can be prepared by adding a solution of xylanase from Streptomyces olivaceoviridis E-86 (47 kDa) by a ratio of 100-500 U xylanase to one gram of carrier Eudergit C. Appropriate amount of enzyme is added to a 0.2-1.2 mol/L phosphate buffer (pH 4.3-7.8), stirring evenly, adding Eudergit C, immobilizing at 4-25° C. for 12-60 h, filtering, and washing to obtain immobilized xylanase. A cold-adaptive β-xylanase XynB form Glaciecola mesophila KMM241 is also suitable for the present invention. This enzyme has an optimum pH of 6.0-7.0 and an optimum temperature of 35° C. Endo-xylanase obtained from Bacillus halodurans is yet another source of enzyme useful in the present invention. β-endoxylanases from Trichoderma sp. K9301 is also suitable for the present invention.

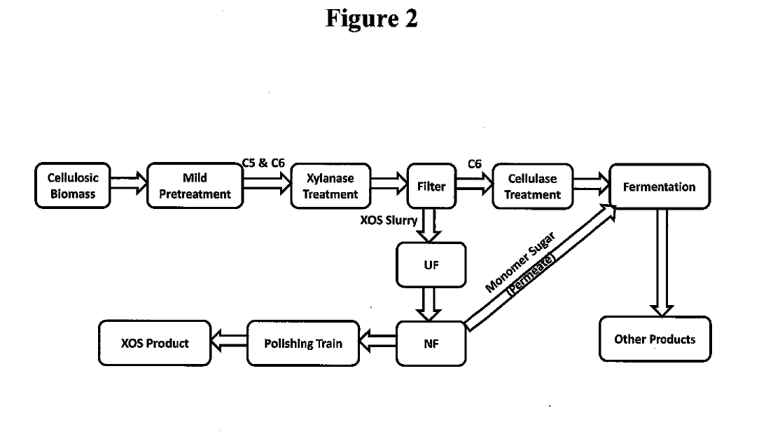

All embodiments of the present invention comprise two hydrolysis steps (FIG. 2). At the end of the first hydrolysis step, the hemicellulose component from the lignocellulosic biomass is recovered either as a monosaccharide or oligosaccharide. In a preferred embodiment, at the end of the first hydrolysis step, the original hemicelluloses component from lignocellulosic biomass is recovered as mixture of oligosaccharides. In a most preferred embodiment of the present invention, at the end of the first hydrolysis step, the original hemicellulose component present in the lignocellulosic biomass is recovered as a highly enriched xylooligosaccharide fraction which can be utilized in human nutrition without much further purification. The aqueous slurry obtained at the end of the first hydrolysis step is subjected to microfiltration process to remove the insoluble materials represented mostly by original cellulose component present in the lignocellulosic biomass. The filtrate from microfiltration contains a mixture of monomeric sugars and xylooligosaccahrides. This filtrate from microfiltration step is further subjected to nanofiltration process to recover xylooligosaccharide in the retendate and fermentable sugar monomers in the filtrate. The contents of xylooligosaccharide and its composition are determined by using ion-exchange chromatography and other appropriate techniques.

The insoluble materials resulting from first hydrolysis step comprising most of the cellulose present in the original lignocellulosic material is subject to a second hydrolysis step. At present, the second round of hydrolysis is catalyzed by enzymes that can collectively hydrolyze cellulose and hemicelluloses to free sugars. In one preferred aspect of this invention, the second hydrolysis step is carried out only with cellulase enzyme so that only glucose is recovered at the end of the second hydrolysis step.

Thermochemical degradation of hemicellulose liberates a number of inhibitors toxic to the fermenting microorganisms. For example, furfurals, 5-hydroxymethyl furfurals and weak acids such as acetic acid, formic acid, and levulinic acid are derived from the thermochemical conversion of hemicellulose. A number of strategies such as neutralization, over liming, activated charcoal, ion-exchange, ethyl acetate+over liming, roto-evaporation, membrane-based separation processes have been developed to remove the fermentation inhibitors. By means of following the two stage hydrolysis, the issues related to inhibitors of cellulase can be eliminated. With the removal of hemicellulose in the first stage with lower amount of acid, the degradation of xylose to furfural that occurs at higher acid concentration can be avoided.

In the second hydrolysis step, the insoluble fraction obtained from first hydrolysis step is first reduced in size so as to increase the surface area and subjected to enzymic hydrolysis. The insoluble fraction resulting from first hydrolysis step is enriched in cellulose and mostly free of xylose, furfural, and hydroxy-methyl furfural. Xylose, furfural, and hydroxy-methyl furfural are inhibitory to the cellulase enzyme used in second hydrolysis step. Thus the advantage of the two-step hydrolysis of the present invention is related to an increased efficiency of cellulase enzyme used in the second step. The insoluble fraction enriched in cellulose is mixed with hydrolytic cellulase enzymes in appropriate containers and maintained at temperature appropriate for the cellulase enzyme activity. In a preferred embodiment, the insoluble fraction form first hydrolysis step is mixed with one or more cellulase enzymes in a conventional rotary cement mixer and maintained at 40-45° C. suitable for cellulase enzyme activity. After specified time of incubation, the second hydrolysis step is terminated and the glucose resulting from the hydrolysis of cellulose is recovered through a suitable fractionation procedure and supplied to the biorefinery operation. Alternately, the simultaneous saccharification and fermentation process can be followed by means of adding suitable biocatalyst at a specified time after the initiation of second hydrolysis step.

At the stage of enzyme hydrolysis of insoluble cellulosic fraction, accessory enzymes such as β-glucosidase, xylanase, and cellulase cofactors, such as GH61, can be added along with cellulase enzymes to considerably enhance the hydrolysis efficiency of cellulase cocktails.

In another embodiment, the pulp material derived from lignocellulosic biomass is used as the raw material for the recovery of XOS. As used in this invention, the term “pulping” refers to the process of chemically or mechanically liberating the individual cellulosic fibers in wood. Kraft cooking process is the predominant pulping process although there are other pulping practices such as sulfite pulping, soda/AQ pulping, solvent pulping, and mechanical pulping. The Kraft process is a chemical pulping process where chipped wood is cooked or digested in a high temperature broth of sodium hydroxide and sodium sulfite cooking liquor. During cooking, lignin and hemicellulose macromolecules are fragmented and solvated, thereby breaking the intercellular adhesive between wood fibers and allowing separation of a pulp extract stream from the cellulose pulp. Kraft and soda pulp mills are energy self-sufficient and often generate excess steam and electricity which can be used by an associated paper mill.

The pulp extract is the source of the hemicelluloses and it is extracted with a caustic solution in a hemicellulose extraction system leading to the production of hemicaustic solution which is an aqueous solution with dissolved hemicellulose. By means of subjecting the hemicaustic solution to the nanofiltration a concentrated hemicellulose solution is obtained. The concentrated hemicelluloses solution is acidified to obtain a paste of highly purified hemicellulose preparation. The highly purified hemicellulose preparation is subjected to enzyme digestion to obtain xylooligosaccahrides.

The cellulose pulp highly enriched in cellulose is subjected to enzyme digestion with cellulase to obtain glucose useful in the biorefinery as a source of organic carbon. The cellulose fraction can be dissolved in an ionic liquid such as 1-butyl-3-methylimidazolium chloride at 130° C. for 2 h and then regenerated with deionized water in order to increase the glucose yield during subsequent enzyme hydrolysis with cellulase enzyme.

In another embodiment of the present invention, the raw lignocellulosic feedstock is subjected to solubilization process prior to pulping. The solubilized hemicelluloses is more readily removed from cellulose fibers than hemicelluloses that has not been pre-hydrolyzed and thereby resulting in accelerated hemicellulose extraction in the hemicelluloses extraction system.

In order to produce food-grade XOS, the resulting liquors have to be refined by removing both monosaccharides and non-saccharide compounds, to obtain a concentrate with an XOS content as high as possible. The usual purity of commercial XOS lies in the range 70-95%. The commercially available XOS syrup or powder have requirement on the degree of polymerization (DP). Typically the 70% XOS syrup has the DP value of X2-4≧50% and X 2-7≧70%. For the 95% XOS powder, the DP values are X 2-4≧65% and X2-7≧95%.

A number of refining strategies such as solvent precipitation, solvent extraction, freeze-drying, and dewaxing can be followed with XOS liquors to get required concentration and purity. Ion exchange chromatography, membrane filtration and activated carbon treatment can also be used for purifying XOS. Nanofiltration can be used to separate xylose from XOS. A combined treatment by nanofiltration, ion exchange and carbon adsorption are effective for cleaning up and concentration of XOS syrup.

One-stage acid hydrolysis of corn cobs with 1% (v/v) sulfuric acid yields fermentable sugars along with various fermentation inhibitors such as furfural, phenolic compounds and acetic acid. The acid hydrolysate can be detoxified using over-liming method or over-liming plus activated charcoal. These detoxification methods are efficient in removing most of the furfural and significant amount of phenolic compounds and acetic acid. Over-liming is done by adding CaO to pH 7.0 and adjusting the pH to 5.0 with sodium sulfite. The activated charcoal treatment is done by adding 3% activated charcoal at 40° C. and shaking at 200 rpm for one hour.

In yet another embodiment of the present invention, the lignocellulosic biomass is subjected to extraction with organic solvent in order to recover lignin in a highly pure form which can be further converted into a number of commercially important compounds and thereby adding to the cost reduction in the operation of biorefinery. The delignified biomass can be subjected to the hydrolytic reactions according to the present invention to recover XOS.

### EXPERIMENTAL SECTION

### General Remarks

Analytical Procedure. The experimental samples generated in this present invention were analyzed following several experimental protocols provided by National Renewable Energy Laboratory (NREL) of the U.S. Department of Energy.

Moisture content, total solids and total dissolved solids in biomass slurry and liquid process samples were determined using the Laboratory Analytical Procedure #102 (LAP-012) issued by NREL on Jul. 5, 1994.

The samples were prepared for compositional analysis following the Laboratory Analytical Procedure entitled “Preparation of Samples for Compositional Analysis” (Technical Report—NREL/TP-510-42620) issued by NREL on Sep. 28, 2005.

Extractives in the various biomass samples of the present invention were determined following the protocols provided in the Laboratory Analytical Procedure entitled “Determination of extractives in Biomass” (Technical Report NREL/TP-510-42619) issued by NREL on Jul. 17, 2005.

Determination of ash content in the various biomass-derived samples of the present invention was carried out following the Laboratory Analytical procedure entitled “Determination of Ash in Biomass” (Technical Report NREL/TP-510-42622) issued by NREL on Jul. 17, 2005.

Determination of structural carbohydrates and lignin in various biomass-derived samples of the present invention was carried out following the Laboratory Analytical Procedure entitled “Determination of Structural Carbohydrates and Lignin in Biomass” (Technical Report NREL/TP-510-42618) issued by NREL on Apr. 25, 2008.

The concentration of Xylose oligomers in various experimental samples were determined using high performance liquid chromatography (HPLC). Samples were neutralized with sodium hydroxide to pH 5.5-7.5. The samples were diluted to 10 mg/ml sugar concentration with deionized (DI) water. Agilent 1100 HPLC apparatus was used with BioRad Aminex HPX-42A column and BioRad Microguard De-Ashing anion and cation guard column. DI water was used as the mobile phase and the flow rate was at 0.6 ml/minute. Refractive Index detector was used at 50° C. Under this HPLC conditions xylotetrose, xylotriose, xylobiose, xylose, fructose, and arabinose were fully-separated allowing accurate quantification of each of these sugar components. All monomer standards were from either Fisher Scientific or Sigma-Aldrich. Xylose oligomer standards were purchased from Megazyme.

Determination of Anion Concentration in Samples Using Ion Chromatography System (ICS).

Dionex 1100 ion chromatography system with Dionex ASRS 300 (4 mm) suppressor, Dionex IonPac AS11-HC column and Dionex IonPac AG11-HC guard column was used for the determination of cation concentration in the samples. 28 mM sodium hydroxide was used as eluent. Approximately 1000 ml of high purity water was added to a 2000 ml volumetric flask, 5.6 mL of 10N sodium hydroxide solution was added to the water in the flask, the total fluid volume in the flask was brought to 2000 ml with high purity water, mixed well by inversion and transferred to eluent bottle in the ICS. A multi-element anion standard was used to generate calibration curves. At least three different calibration standards (20, 10 and 1 ppm) were used to establish a calibration curve for each ion. Liquid samples for analysis were diluted using deionized water and filtered through a 0.2 μm filter. Solid samples were dissolved in appropriate volumes of deionized water and filtered through 0.2 μm filter. The following parameters were used in running the ICS. Flow rate: 1.5 mL/minute; column temperature: 30° C.; Cell temperature: 35° C.; Suppressor current: 104 mA; Sample delivery speed: 4 ml/minute; Analysis time: 13 minutes; Delay volume: 125 ml; Flush factor: 5; Data Collection Rate: 5 Hz.

Determination of Cation Concentration in Samples Using Ion Chromatography System (ICS).

Dionex 1100 ion chromatography system with Dionex CSRS 300 (4 mm) suppressor, Dionex IonPac CS16-HC column and Dionex IonPac CG16-HC guard column was used for the determination of cation concentrations in the samples. Standards should have a known purity in order to accurately calculate the cation concentrations in the sample. Using concentrated standards, working standards were prepared. For example, by means of dissolving 2.5 mL of 1000 ppm standard to 50 mL of deionized water, a working standard of 50 ppm was prepared. Cation standards can all be combined into one working standard. 35 mM methanesulfonic acid was used as eluent. Approximately 1000 ml of high purity water was added to a 2000 ml volumetric flask, 5.76 g of concentrated methanssulfonic acid solution was transferred to the water in the flask, the total fluid volume in the flask was brought to 2000 ml with high purity water, mixed well by inversion and transferred to eluent bottle in the ICS. All liquid samples for testing were diluted with deionized water and filtered through 0.2 μm filter. The solid samples were diluted with deionized water and filtered through 0.2 μm filter. The following parameters were followed in running the ICS. Flow Rate: 1.0 mL/minute; Column Temperature: 40° C.; Cell Temperature: 45° C.; Suppressor Current: 103 mA; Analysis Time: 19 minutes; Sample Deliver Speed: 4 mL/minute; Delay Volume: 125 μL; Flush Factor: 5; Data Collection Rate: 5 Hz. Control sample and blank sample were run after every 10 injections and at the completion of a run to account for any possible drift. Samples and controls were integrated to calculate the results.

Organic acid and alcohols were analyzed using an Agilent 1200 HPLC with the following parameters: Mobile phase: 0.008N sulfuric acid; Column: BioRad Aminex HPX-87H; Guard column: BioRad Microguard Cation H+, column Temperature: 50° C.; run time: 55 min; Detectors: UV: 210 nm; RI 45° C.; Flow Rate: 0.6 mL/min. A check of the hydroxymethyl furfural (HMF) and furfural was performed using an HPLC method with C18 column and water/methanol gradient and a UV detector set to 278 nm.

### Example 1

### Composition of Corn Cob Materials

Two different types of corn cobs namely Corn Cob 814 and Corn Cob 1014 used in the present invention (Grit-O'Cobs 2040) were obtained from Andersons, Inc. (Maumee, Ohio 43537, USA) and their chemical composition as presented in the Table 1 was determined using appropriate experimental protocols provide in the section above. On an average, the corn cobs used in the present invention for the recovery of economically-valuable xylooligosaccharides contained about 30% of xylans on a dry weight basis.

### Example 2

### Recovery of Xylooligosaccharides Form Corn Cob

Corn cob grits were subjected to mechanical milling to obtain particles of appropriate size. The objective of particle reduction was to get a suitable particle size so that there is no dust explosion in the plant and the solid/liquid separation is achieved without much difficulty.

After appropriate size reduction, the corn cob grit was impregnated with dilute sulfuric acid (0.1%, i.e. 1 g/L) either at 60° C. overnight (12 h) or at 100° C. for shorter duration of 45 minutes to 90 minutes in a steaming impregnator. The impregnation experiments were done in batches. In each impregnation experiments, 1 kg of size-reduced corn cob was mixed with 9 liters of 0.1% sulfuric acid and incubated at specified temperatures for specified time. At the end of the impregnation the slurry was tested for the release of sugars, ions, HMF, furfural and lignin. The lignin release was monitored by measuring the absorbance of the aqueous phase of the impregnation sample at 320 nm. The results of the analysis of various corn cob samples after impregnation with sulfuric acid for specified period of time at specified temperature are shown in the Table 1. As the results shown in Table 2 illustrates, the impregnation at 100° C. for a period of 45 minutes to 90 minutes was effective in releasing more of chlorine ions and lignin material from the corn cob as compared to the impregnation treatment with sulfuric acid at 60° C. overnight. Since there is a definite advantage in impregnating the corn cob material at elevated temperature for a shorter period of time, the impregnation treatment at 100° C. for a period of 45 minutes to 90 minutes was used in the subsequent experiments and is considered as the preferred embodiment for the impregnation process with sulfuric acid according to the present invention.

After specified duration of impregnation with sulfuric acid at specified temperature, sulfuric acid was discharged from the steaming incubator and the remaining solid material was washed three times with equal amount of deionized water and the deionized water was drained out and the resulting semidry material was loaded into a 10 L Parr reactor for pretreatment. The term “pretreatment” as used in this present invention refers to the process by which the corn cob materials impregnated with dilute sulfuric acid is subjected to thermochemical hydrolysis leading to the release of xylooligosaccharides which can be recovered through subsequent downstream processing steps.

About 2.5-3 kg of semidry material with about 60% water content recovered from impregnation step was loaded into a Parr reactor. The temperature of the Parr reactor was increased to 80° C. by means of heating the jacket of the Parr reactor through steam circulation. Once the temperature of the Parr reactor reached, the temperature of corn cob material inside the Parr reactor was further raised through direct steam injection. This direct steam injection besides increasing the temperature further, elevated the pressure within the Parr Reactor. The temperature within the Parr reactor was varied from 140° C. to 163° C. and the pressure is kept in the range of 75 pounds per square inch (psi) to 145 psi. The duration of this thermochemical pretreatment was also varied from 20 minutes to 30 minutes. At the end of the pretreatment with direct steam injection, the volume of the corn cob material within the Parr reactor increased to 5.0 L to 0.6.0 L (075) Pretreatment was carried out with both corn cob 1014 and corn cob 814. Immediately after pretreatment, each sample was subjected to chemical analysis for the release of glucose, xylose, arbinose, acetic acid, HMF, furfural, potassium, chloride, phosphate, and lignin as measured by absorbance at 320 nm (Tables 3 and 4).

The pretreated corncobs were cooled downed to 50° C. and the pH was adjusted to 5-5.3 with the addition of CaO/Ca(OH)₂ or ammonia. Endo-xylanase enzyme HTec2®, from Novazymes® was added at the dose of 1.0 g enzyme/100 g of dry corncobs. The enzymic hydrolysis continued for a period 48 hours and samples were removed at 0, 3, 6, 24, 30 and 48 hours for measuring the amount of xylooligosaccahride released from impregnated and pretreated corn cob upon digestion with endo-xylanase.

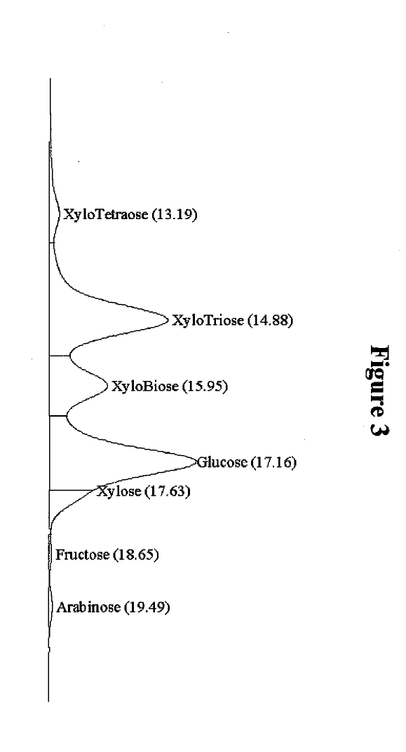

The HPLC method used in the present invention was able to clearly resolve the peaks attributable to several different sugars released from corn cob due to endo-xylanase treatment as shown in FIG. 3. Table 5 provides the amount of xylotetrose, xylotriose, xylobiose and xylose that were released during 48 hours of incubation with enzyme HTec2®, Table 6 provides the amount of glucose, fructose and arabinose that were releases during 48 hours of incubation with the enzyme HTec2®.

Table 7 provides the data on the XOS yield upon digestion of various pretreated corn cob samples upon digestion with the enzyme HTec2®. Provided in this table are the volume of the pretreated corn cob material upon digestion with the endo-xylanase enzyme and the total XOS concentration in the pretreated corn cob material after enzyme digestion. The total XOS concentration is the sum of xylotetrose, xylotriose and xylobiose as determined by HPLC analysis. From the total volume of the pretreated and enzyme-digested corn cob material and the XOS concentration derived from HPLC analysis, the total yield of XOS from the enzyme digestion was calculated and shown on the fourth column of the Table 7. Also shown in the Table 7 is the theoretical yield of the XOS that can be derived from the corn cob material that was subjected to impregnation, pretreatment and enzyme digestion. The actual content of the xylans in the starting corn cob material is taken into consideration in calculating the theoretical yield of the XOS shown in the fifth column of the Table 7. As shown in the Table 1, the starting corn cob materials contain about 30% xylans on a dry weight basis. This determination of the xylan content in the starting corn cob material, the amount of corn cob material used in the impregnation, pretreatment and enzyme treatment and the final volume of the corn cob material after enzyme treatment, the theoretical yield of XOS at the end of the enzyme is calculated with the assumption that the enzyme treatment after pretreatment would release all of the xylan present in the starting corn cob material as XOS. This calculated theoretical yield for XOS and the actual XOS concentration detected in the enzyme-treated corn cob material, the percentage yield for XOS was calculated for each of pretreated samples as shown in the sixth column in the Table 7. In essence, the Table 7 compares the XOS yield resulting from several different pretreatment processes. As shown in Table 7, maximum yield of XOS (87.16 percentage yield) was obtained by using the pretreatment at 75 psi and 140° C. for a period of 30 minutes. A harsher pretreatment involving higher temperature and higher pressure yielded a lower percentage for XOS. This lower yield was likely to be degradation of XOS into xylan monomers under the harsher condition of high pressure and high temperature.

### Example 3

### Downstream Processing of the XOS Recovered after Pretreatment and Enzyme Digestion

After enzyme treatment, the resulting slurry was subjected to a series of downstream processing steps to recover XOS in a concentrated form with a reduced amount of xylose and other monomeric sugars. In the first of this lengthy downstream processing, the slurry resulting from enzyme digestion was pressed through a cheese cloth to remove remaining corn cob grits. The filtrate from this initial coarse filtration was subjected to filtration in a hydraulic press. The output stream from hydraulic press was fed into a filter press to remove fines using about 5 micron filter pads. The output from the filter press was fed into a Millipor microfiltration unit with a 0.22 μm Pellicon cassette. The output stream from microfiltration unit was fed into a Millipore ultrafiltration unit with a10kD Pellicon cassettes having a filtration area of 1 square meter. Table 8 provide the details about the mass of the corn cob-derived material that was fed into each of the downstream filtration units and the amount of XOS and monomers in the output stream form each of the filtration units.

The output stream form ultrafiltration unit was used as a feed for the nanofiltration unit. Nanofiltration was conducted on the GEA skid using NF-245 spiral membrane. As shown in Table 9, three different permeate fractions were collected from nanofiltration unit with minimal amount of XOS and significant levels of monomers suggesting that the XOS is retained within the nanofiltration unit with a cut off of 300 daltons. The retentate pool in the nanofiltration unit was concentrated about 4× before two diafiltrations were carried out using 10 L of DI water each. The goal of the diafiltration was to remove impurities and push more monomer sugars into the permeate stream. Table 9 provides the concentration of XOS in the retentate of the nanofiltration unit before and after diafiltration. Also shown in Table 9 is the value for “yellow index” in various fractions after nanofiltration.

The XOS fraction from nanofiltration unit is further subjected to batch carbon treatment and polishing step to further improve the quality of the XOS preparation. The improvement in the quality of XOS is accompanied by a decrease in the yellow index as shown in the Tables 10 and 11.

The polishing step involved the passage of the XOS enriched fraction through four different columns to remove impurities contributing the dark coloration of the XOS enriched fraction. In the first step, the XOS fraction is treated with (2.5-10%) activated carbon at 50° C. for a period of 3-6 hours. In the next stage, the XOS containing material was passed through Lanxess Lewatit MonoPlus S 108H cation resin at the flow rate of 3 bed volumes per hour. This step was followed by a passage through a column containing LanXess A365 anion resin at the flow rate of 3 bed volume per hour. Finally, the XOS enriched fraction was passed through a column containing activated Calgon Carbon CPG at the flow rate of 3 bed volumes per hour. Following the polishing step, the XOS preparation was evaporated in a Rotovap to further concentrate the XOS syrup.

Table 12 provides the chemical composition of the XOS fraction obtained at the end of the polishing step of the present invention.

### REFERENCES

U.S. Pat. No. 5,340,403
U.S. Pat. No. 5,536,325
U.S. Pat. No. 5,939,309
U.S. Pat. No. 6,692,578
U.S. Pat. No. 6,752,902
U.S. Pat. No. 6,838,099.
U.S. Pat. No. 6,942,745
U.S. Pat. No. 6,942,754
U.S. Pat. No. 7,812,153
U.S. Pat. No. 7,842,490
U.S. Patent Application Publication No. US 2002/0195213 A1
U.S. Patent Application Publication No. US 2008/0202504 A1
U.S. Patent Application Publication No. US 2009/0062232 A1
U.S. Patent Application Publication No. US 2009/0308383.A1
U.S. Patent Application Publication No. US 2010/0263814 A1
U.S. Patent Application Publication No. US 2011/0129886 A1
International Patent Application Publication No. WO 2008/021141 A2
International Patent Application Publication No. WO 2010/115067 A2
International Patent Application Publication No. WO 2011/066487 A1
Aachary A A., Prapulla S C. (2011) Xylooligosaccharides (XOS) as an emerging prebiotic: microbial synthesis, utilization, structural characterization, bioactive, properties, and applications. Comprehensive reviews in food science and food safety. 10:2-16.
Akpinar, O., Ak, O., Kavas, A., Bakir, U. and Yilmaz, L. (2007) Enzymatic production of xylooligosaccahrides from cotton stalks. J. Agric. Food Chem. 55: 5544-5551.
Akpinar O., Erdogan K., Bostanci S. (2009) Production of xylooligosaccharides by controlled acid hydrolysis of lignocellulosic material. Carbohydrate Research. 344: 660-666.
Akpinar, O., Erdogan, K. and Bostanci, S. (2009) Enzymatic production of xylooligosaccharide from selected agricultural wastes. Food Bioproducts Proc. 87: 145-151.
Amidon, T. E., Bujanovic, B., Liu, S. and Howard, J. R. (2011) Commercializing biorefinery technology: A case for the multi-product pathway to a viable biorefinery. Forest 2: 929-947.
Cana, A. and Palet, C. (2007) Xylooligosaccharide recovery from agricultural biomass waste treatment with enzymatice polymeric membranes and characterization of products with MALDI-TOF-MS. J. Memb. Sci. 291:96-105.
Caparros, S., Garrote, G., Ariza, J., Diaz, M. J. and Lopez, F. (2007) Xylooligosaccharides production from Arundo donax. J. Agric. Food Chem. 55: 5536-5543
Chen, L-L., Zhang, M., Zhang, D-H., Chen, X-L., Sun, C-Y., Zhou, B-C. and Zhang, Y-Z. (2009) Purification and enzymatice characterization of two b-endoxylanases from Trichoderma sp. K9301 and their actions in xylooligosaccharide production. Bioresource Technol. 100: 5230-5236
Ge, J-P., Cai, B-Y., Liu, G-M., Ling, H-Z., Fang, B-Z., Song, G., Yang, X-F. and Ping, W-X. (2011) Comparison of different detoxification methods for corn cob hemicellulose hydrolysate to improve ethanol production by Candida shehatae ACCC 20335. African J. Microbiol. Res. 5: 1163-1168.
Geng, X. and Henderson W. A. (2012) Pretreatment of corn stover by combining ionic liquid dissolution with alkali extraction. Biotechnol. Bioeng. 109: 84-91
Goldman, N. (2009) Methods for optimizing enzymatic hydrolysis of xylan to improve xylooligosaccahride yield. Basic Biotechnol. 5: 31-36.
Hu, J., Arantes, V. and Saddler, J. N. (2011) The enhancement of enzymatic hydrolysis of lignocellulosic substrates by the addition of accessory enzymes such as xylanse: is it an additive or synergistic effect? Biotechnol. Biofuel. 4: 36-49.
Jantama, K., Haupt, M. J., Svoronos, S. A., Zhang, X., Moore, J. C., Shanmugham, K. T. and Ingram, L. O. (2008a) Combining metabolic engineering and metabolic evolution to develop nonrecombinant strains of Escherichia coli C that produce succinate and malate. Biotechnol. Bioeng. 99: 1140-1153.
Jantama, K., Zhang, X., Moore, J. C., Shanmugam, K. T., Svoronos, S. A. and Ingram, L. O. (2008b) Eliminating side products and increasing succinate yields in engineered strains of Escherichia coli C. Biotechnol. Bioeng. 101: 881-893.
Kumar, D. and Murthy, G. S. (2011) Impact of pretreatment and ownstream processing technologies on economics and energy in cellulosic ethanol production. Biotechnol. Biofuels. 4: 27-46
Taherzadeh, M. J. and Karimi, K. (2008) Pretreatment of lignocellulosic wastes to improve ethanol and biogas production: A review. Int. J. Mol. Sci. 9: 1621-1651
Vegas R., Luque S., Alvarez J R., Alonson J L, Dominguez H., Parajo J C. (2006) Membrane-Assisted Processing of Xylooligosaccharide-Containing Liquors. J. Agric. Food Chem. 54:5430-5436.
Vazquez, M. J., Alonso, J. L., Dominguez, H. and Parajo, J. C. (2000) Xylooligosaccharides: manufacture and applications. Trends in Food Science & Technology. 11: 387-393.
Vazquez M. J., Garrote G. Alonso J. L., Dominguez H., Parajó J. C. (2005) Refining of antohydrolysis liquors for manufacturing xylooligosaccharides: evaluation of operational strategies. Bioresource Technol. 96:889-896.
Wang, J., Sun, B., Cao, Y., Tian, Y. and Wang, C. (2009) Enzymatice preparation of wheat bran xylooligosaccharides and their stability during pasteurization and autoclave sterilization at low pH. Carbohydrate Polymers. 77: 816-821.
Wang B., Chen B., Feng H. (2008) Enrich arabinoxylan in corn fiber for value-added products. Biotechnology Letter. 30: 275-279.
Wang Y. (2009) Prebiotics: Present and future in food science and technology. Food Research International. 42: 8-12.
Wang, Z., Xu, J. and Cheng, J. J. (2011) Modeling biochemical conversion of lignocellulosic materials for sugar production: a review. BioResources 6: 5282-5306.
Zhang, M., Su, R., Qi, W. and He, Z. (2010) Enhanced enzymatic hydrolysis of lignocelluloses by optimizing enzyme complexes. All. Biochem. Biotechnol. 160: 1407-1414.
Zhang, L., Li, J-H., Li, S-Z. and Liu, Z. L. (2011) Challenges of cellulosic ethanol production form xylose-extracted corncob residues. Bioresources 6: 4302-4316.
U.S. Billion-ton Update—Biomass Supply for a Bioenergy and Bioproducts Industry. A report prepared by Oak Ridge National Laboratory, Oak Ridge, Tenn., USA for United States Department of Energy (August 2011).

TABLE 1
Composition of corn cob materials

| Component (weight %) | Corn Cob 814 | Corn Con 1014 |
|---|---|---|
| Glucan | 42.28 | 40.06 |
| Xylan | 29.08 | 33.57 |
| Arabinan | 2.06 | 2.39 |
| Moisture | 7.88 | 7.73 |
| Ash | 1.46 | 1.62 |
| Water Extract | 6.55 | 7.73 |
| Ethanol Extract | 0.63 | 1.07 |
| AIR | 15.98 | 13.50 |
| AIL | 15.93 | 13.57 |
| ASL | 0.02 | 0.02 |
| Total Lignin | 15.95 | 13.59 |
| Lignin (Raw) | 14.81 | 12.39 |
| Acetate | 3.10 | 3.00 |

TABLE 2
Comparison of impregnation conditions

| | Impregnation Conditions | | | | | |
|---|---|---|---|---|---|---|
| Chemical | 60 C. | 100 C. | 100 C. | 100 C. | 100 C. | 100 C. |
| Component | 12 h | 1.5 h | 45 min | 1 h | 1 h | ih |
| Glucose | 0.55 | 3.65 | 2.64 | 3.18 | 3.07 | 1.95 |
| (g/L) | | | | | | |
| Xylose | — | — | — | — | — | — |
| (g/L) | | | | | | |
| Potassium | 207.2 | 282.7 | 190.3 | 262.3 | 280.9 | 238.8 |
| (mg/L) | | | | | | |
| Chloride | 56.25 | 264.2 | 186.7 | 262.9 | 245.3 | 226.9 |
| (mg/L) | | | | | | |
| HMF | — | — | — | — | — | — |
| (g/L) | | | | | | |
| Furfural | — | — | — | — | — | — |
| (g/L) | | | | | | |
| 320 nm | 1.34 | 15.68 | 11.84 | 9.46 | 9.74 | 5.97 |
| Abs | | | | | | |

TABLE 3
Effect of pretreatment of corn cob 1014

| | Pretreatment conditions | | | | | | | |
|---|---|---|---|---|---|---|---|---|
| | Pressure (psi)/Temperature (° C.)/Duration (min) | | | | | | | |
| | 145/ | 150/ | 110/ | 100/ | 90/ | 80/ | 80/ | 75/ |
| | 163/ | 165/ | 157/ | 154/ | 145/ | 142/ | 142/ | 140/ |
| Component | 25 | 20 | 20 | 20 | 30 | 30 | 45 | 30 |
| pH | 2.9 | 2.7 | 2.7 | 2.7 | 2.76 | 2.76 | 2.66 | 2.97 |
| Glucose | 0.61 | 0.57 | 0.37 | 0.34 | 0.25 | 0.63 | 0.63 | 0.12 |
| (g/L) | | | | | | | | |
| Xylose | 16.8 | 30.6 | 23.4 | 18.6 | 12.7 | 7.14 | 16.4 | 6.05 |
| (g/L) | | | | | | | | |
| Arabinose | 2.33 | 3.1 | 3.0 | 2.7 | 2.49 | 2.65 | 2.6 | 2.21 |
| (g/L) | | | | | | | | |
| Acetic acid | 1.53 | 2.2.4 | 1.58 | 1.27 | 1.26 | 0.83 | 3.04 | 0.81 |
| (g/L) | | | | | | | | |
| HMF | 0.08 | 0.05 | 0.02 | 0.02 | — | — | 0.03 | — |
| (g/L) | | | | | | | | |
| Furfural | 1.18 | 1.48 | 0.77 | 0.55 | — | 0.20 | 0.92 | — |
| (g/L) | | | | | | | | |
| Potassium | 93.3 | 7.15 | 3.95 | 30.5 | 2.59 | 9.60 | 2.1 | 13.9 |
| (mg/L) | | | | | | | | |
| Chloride | 22.3 | 2.03 | 2.21 | 6.5 | 4.21 | 1.68 | 0.7 | 1.6 |
| (mg/L) | | | | | | | | |
| Phosphate | — | — | — | — | 21.3 | 336.04 | 9.56 | 17.1 |
| (mg/L) | | | | | | | | |
| 320 nm abs | 216 | 252 | 260 | 232 | 271 | 246 | 224 | 251 |

TABLE 4
Pretreatment of corn cob 814

| | Pretreatment conditions | | | |
|---|---|---|---|---|
| | Pressure (psi)/Temperature (° C.)/Duration (min) | | | |
| | 75/ | 75/ | 75/ | 75/ |
| | 140/ | 140/ | 140/ | 140/ |
| Component | 30 | 30 | 30 | 30 |
| pH | 2.7 | 2.89 | 2.84 | 2.90 |
| Glucose (g/L) | 0.11 | 0.10 | 0.13 | 0.08 |
| Xylose (g/L) | 5.0 | 4.89 | 5.07 | 5.50 |
| Arabinose (g/L) | 2.11 | 2.04 | 2.26 | 1.86 |
| Acetic acid (g/L) | 0.71 | 0.42 | 0.48 | 0.59 |
| HMF (g/L) | — | — | — | — |
| Furfural (g/L) | 0.14 | 0.11 | — | 0.14 |
| Potassium (mg/L) | 11.9 | 13.17 | 15.21 | 7.38 |
| Chloride (mg/L) | 1.9 | 1.9 | 9.3 | 7.38 |
| Phosphate (mg/L) | 18.1 | — | 53.0 | 1.4 |
| 320 nm abs | 212.7 | 257.3 | 210.0 | 214.5 |

TABLE 5
Enzyme treatment of pretreated corn cob

| | Time | Xylotetrose | Xylotriose | Xylobiose | Xylose |
|---|---|---|---|---|---|
| Sample | (h) | (g/L) | (g/L) | (g/L) | (g/L) |
| 1A | 0 | 1.14 | 6.44 | 11.10 | 15.52 |
| 1A | 3 | 0.76 | 7.47 | 11.41 | 15.14 |
| 1A | 6 | 0.88 | 8.46 | 13.07 | 16.51 |
| 1A | 24 | 0/67 | 11.36 | 13.75 | 17.34 |
| 1A | 30 | 0/79 | 12.40 | 13.75 | 17.27 |
| 1A | 48 | 0.86 | 14.15 | 13.86 | 17.36 |
| 1B | 0 | 0.98 | 5.96 | 44.71 | 14.80 |
| 1B | 3 | 0.94 | 6.97 | 11.84 | 15.07 |
| 1B | 6 | 0.80 | 7.57 | 12.55 | 16.12 |
| 1B | 24 | 0.65 | 12.93 | 14.00 | 17.49 |
| 1B | 30 | 0.75 | 13.48 | 14.09 | 17.69 |
| 1B | 48 | 0.98 | 14.87 | 14.32 | 17.05 |

TABLE 6
Enzyme treatment of pretreated corn cob

| | Time | Glucose | Fructose | Arabinose |
|---|---|---|---|---|
| Sample | (h) | (g/L) | (g/L) | (g/L) |
| 1A | 0 | 4.50 | 1.07 | 1.78 |
| 1A | 3 | 3.99 | 0.23 | 0.90 |
| 1A | 6 | 5.69 | 0.92 | 1.75 |
| 1A | 24 | 7.11 | 0.92 | 1.72 |
| 1A | 30 | 8.22 | 0.92 | 1.71 |
| 1A | 48 | 9.27 | 0.92 | 1.71 |
| 1B | 0 | 4.58 | 0.93 | 1.78 |
| 1B | 3 | 4.99 | 0.92 | 1.74 |
| 1B | 6 | 5.02 | 0.92 | 1.73 |
| 1B | 24 | 8.62 | 0.94 | 1.75 |
| 1B | 30 | 9.06 | 0.93 | 1.75 |
| 1B | 48 | 11.66 | 0.93 | 1.77 |

TABLE 7
XOS yield from pretreated corn cob upon enzymatic treatment

| Pretreatment | XOS | Vol- | XOS | Theoretical | |
|---|---|---|---|---|---|
| condition | conc. | ume | Yield | Yield | % |
| (psi/C./min) | (g/L) | (L) | (g) | (g/L) | Yield |
| 145 psi/163 C./25 min | 29.52 | 4.8 | 141.7 | 69.94 | 42.21 |
| 150 psi/165 C./20 min | 31.135 | 5.2 | 161.9 | 64.56 | 48.23 |
| 150 psi/165 C./20 min | 26.57 | 4.8 | 127.54 | 69.94 | 37.99 |
| 150 psi/165 C./20 min | 23.91 | 5.35 | 127.92 | 62.75 | 38.11 |
| 110 psi/157 C./20 min | 33.195 | 5.2 | 172.6 | 64.56 | 51.42 |
| 110 psi/157 C./20 min | 33.45 | 5.5 | 183.96 | 61.04 | 54.80 |
| 100 psi/154 C./20 min | 29.62 | 6 | 177.72 | 55.95 | 52.94 |
| 90 psi/145 C./30 min | 33.65 | 5.9 | 198.54 | 56.90 | 59.14 |
| 80 psi/142 C./30 min | 39.5 | 4.3 | 169.85 | 78.07 | 50.60 |
| 80 psi/142 C./45 min | 34.9 | 4.2 | 146.58 | 79.93 | 43.66 |
| 80 psi/142 C./30 min | 35.23 | 5.2 | 183.196 | 64.56 | 54.57 |
| 80 psi/142 C./30 min | 39.81 | 2.6 | 103.506 | 64.56 | 61.67 |
| 75 psi/140 C./30 min | 37.99 | 5.55 | 210.845 | 60.49 | 62.82 |
| 75 psi/140 C./30 min | 44.655 | 4.6 | 205.413 | 72.98 | 61.19 |
| 75 psi/140 C./30 min | 38.435 | 4.6 | 176.801 | 72.98 | 52.67 |
| 75 psi/140 C./30 min | 44.535 | 4.5 | 200.41 | 74.60 | 59.70 |
| 75 psi/140 C./30 min | 45.62 | 4.5 | 205.29 | 74.60 | 61.15 |
| 75 psi/140 C./30 min | 59.65 | 4.5 | 268.425 | 74.60 | 79.96 |
| 75 psi/140 C./30 min | 49.71 | 5.4 | 268.434 | 62.17 | 79.96 |
| 75 psi/140 C./30 min | 51.33 | 5.7 | 292.581 | 58.89 | 87.16 |

TABLE 8
Downstream processing of corn cob after
pretreatment and enzymatic hydrolysis

| Stage in | Mass | XOS | Monomers |
|---|---|---|---|
| downstream processing | (kg) | (g/L) | (g/L) |
| Output from Hydraulic Press | 73.3 | 44.97 | 25.89 |
| Output from Filter Press | 58.17 | 41.39 | 25.58 |
| Output from microfiltration | 57.00 | 32.76 | |
| Output from ultrafiltration | 53.183 | 33.0 | 1.64 |

TABLE 9
Nanofiltration of XOS containing sample

| | Mass | XOS | Monomer Sugars | |
|---|---|---|---|---|
| Sample | (kg) | (g/L) | (g/L) | YI |
| Feed Pool | 53.183 | 33.0 | 1.64 | 73.3 |
| Permeate # 1 | 19.5 | 0.63 | 11.32 | 8.92 |
| Permeate #2 | 19.5 | 1.4 | 14.94 | 8.92 |
| Permeate # 3 | 19.5 | 1.88 | 22.33 | 8.92 |
| Retentate before | 10 | 126.48 | 58.79 | n/a |
| diafiltraton | | | | |
| Retentate after | 14.514 | 117.97 | 32.66 | 127.67 |
| diafiltration | | | | |

TABLE 10
Batch Carbon Treatment

| Sample | Mass (kg) | XOS (g/L) | YI |
|---|---|---|---|
| Pool from nanofiltration | 14.514 | 117.97 | 127.67 |
| Batch Carbon Treated #1 | 7.209 | 87.944 | 34.92 |
| Batch Carbon Treated # 2 | 7.305 | 88.52 | 34.92 |

TABLE 11
Polishing train

| Sample | Mass (kg) | XOS (g/L) | YI |
|---|---|---|---|
| Feed Pool | 13.023 | 88.236 | 34.92 |
| Production Pool | 11.385 | 94.64 | 7.04 |

TABLE 12
Chemical composition of XOS preparation

| Component/Property | Amount/Measurement | |
|---|---|---|
| >X4 | None | |
| Xylotetrose | 1.2% | (w/w) |
| Xylotriose | 19.0% | (w/w) |
| Xylobiose | 29.5% | (w/w) |
| Total X2-X4 | 49.70% | (w/w) |
| Xylose | 5.6% | (w/w) |
| Glucose | 9.7% | (w/w) |
| Arabinose | 0.6% | (w/w) |
| Frucotse | 0.5% | (w/w) |
| Chloride | 18.25 | (mg/kg) |
| Sulfate | 4.87 | (mg/kg) |
| Ammonium | 2.07 | (mg/g) |
| Cu | 0.60 | (mg/g) |
| Fe | 0.82 | (mg/g) |
| K | 1.10 | (mg/g) |
| Na | 6.82 | (mg/g) |

| | |
|---|---|
| YI | 46.2 |
| APHA | 900 |
| Density (g/ml) | 1.314 |

What is claimed is:

1. A method for hydrolyzing lignocellulosic material, comprising the steps of:
(a) subjecting the lignocellulosic material to pretreatment step in an aqueous medium at a temperature and pressure optimal for depolymerization of hemicellulose to produce a slurry without any noticeable impact on the cellulose and lignin components in the lignocellulosic material;
(b) optionally subjecting the pretreated lignocellulosic material to an enzyme hydrolysis step
(c) subjecting the slurry resulting from step (a) to a first separation process to obtain an aqueous phase containing products resulting from the depolymerization of hemicellulose in step (a) and undissolved material containing cellulose and lignin;
(d) processing the aqueous phase obtained in step (c) to recover xylooligosaccharide from xylose monomer;
(e) subjecting the undissolved material resulting from step (c) to a second hydrolysis step under conditions that facilitates the depolymerization of cellulose to a slurry comprising glucose; and
(f) subjecting the slurry resulting from step (e) to a second separation process to obtain an aqueous phase containing glucose and a solid fraction comprising primarily of lignin.

2. The Process according to claim 1, wherein said lignocellulosic material is selected from a group consisting of agricultural wasters, forestry waste, municipal waste and energy crops.

3. The process according to claim 1, wherein said lignocellulosic material is a hardwood or a softwood.

4. The process according to claim 1, wherein said lignocellulosic material is selected from corn stover or corn cobs.

5. The process according to claim 1 wherein the process further comprises a mild enzyme treatment immediately after pretreatment step.

6. The process according to claim 1, wherein the process further comprises a step of subjecting the aqueous phase obtained from the first separation process to the action of endoxylanase enzyme to produce xylooligosaccharide.

7. The process according to claim 1 where in the xylooligosaccharide comprises other monomeric sugar residues.

8. The process according to claim 1 wherein the process further comprising a step of dissolving the insoluble fraction form step (b) in an ionic liquid and regenerating the insoluble fraction with deionized water.

9. A method for hydrolyzing lignocellulosic material comprising the steps of:
(a) providing cellulose pulp that has hemicellulose that is predominantly xylan, and a lignin content that is less that 1 wt %;
(b) extracting the hemicellulose from the pulp as in step (a) into caustic solution thereby forming a hemicaustic solution and a washed pulp;
(c) separating the hemicaustic solution into a concentrated hemicellulose solution and concentrated caustic solution;
(d) acidifying concentrated hemicellulose solution and recovering hemicellulose in a concentrated form;
(e) subjecting hemicellulose recovered in step (d) to enzymatic hydrolysis to produce xylooligosaccahrides; and
(f) subjecting the washed pulp obtained in step (b) to enzymatic hydrolysis to produce glucose.

10. The method for hydrolyzing lignocellulosic material as in claim 9, wherein the pulp is derived from solubilized lignocellulosic material.
